(19) United States
(12) Patent Application Publication
(10) Pub. No.: US 2024/0032808 A1
(43) Pub. Date: Feb. 1, 2024

(54) DEVICE AND METHOD FOR MEASURING AN ARTERIAL PRESSURE

(71) Applicant: Commissariat à l'Energie Atomique et aux Energies Alternatives, Paris (FR)

(72) Inventors: Stéphane BONNET, Grenoble Cedex 09 (FR); Xavier BEDNAREK, Grenoble Cedex 09 (FR)

(73) Assignee: Commissariat à l'Energie Atomique et aux Energies Alternatives, Paris (FR)

(21) Appl. No.: 18/362,014

(22) Filed: Jul. 31, 2023

(30) Foreign Application Priority Data
Jul. 31, 2022 (FR) 22 07947

(51) Int. Cl. A61B 5/0225; A61B 5/00; A61B 5/02; A61B 5/022

(52) U.S. Cl. CPC A61B 5/0225; A61B 5/7278; A61B 5/02007; A61B 5/02233; A61B 2560/0223

(57) ABSTRACT

The invention relates to a method for determining an arterial pressure of a user, by measuring a physiological parameter, the physiological parameter passing through an extremum when the transmural pressure of the artery is zero, the method comprising:
a) applying a pressure to the artery, so as to modify the transmural pressure of the artery;
b) measuring the physiological parameter of the user by means of a sensor;
c) establishing a calibration function, the calibration function defining a relationship between the transmural pressure and the parameter;
d) applying a pressure to the artery at a measurement time, and measuring the physiological parameter at the measurement time;
e) estimating a transmural pressure at the measurement time;
f) based on the transmural pressure estimated in step e) and on the pressure applied at the measurement time, estimating an arterial pressure of the user.

### TECHNICAL FIELD

The technical field of the invention is measurement of arterial pressure.

### PRIOR ART

Most devices allowing arterial pressure to be measured use a pressure sensor coupled to a compression cuff placed on a limb, generally an arm. Arterial pressure is characterized by measuring the pressure exerted by the cuff at one or more characteristic times. The pressure sensor is sensitive to the beats of the heart and to their amplitude. In general, these devices are placed around the brachial artery.

In mass-market blood-pressure monitors, a pressure sensor determines the air pressure in the cuff. The cuff is compressed so as to obtain arterial occlusion. During deflation or inflation of the cuff, pressure oscillations appear. These oscillations increase until, transiently, a maximum amplitude is reached. At this time the pressure in the cuff is considered equal to the mean arterial pressure in the brachial artery. Based on the detected maximum amplitude, the times corresponding to the systolic and diastolic arterial pressures are estimated using empirical laws. It is conventionally considered that:
the time corresponding to the systolic arterial pressure is prior to the time at which the mean arterial pressure is reached, and corresponds to an amplitude of oscillation of a certain percentage of the maximum amplitude, 55% for example;
the time corresponding to the diastolic arterial pressure is subsequent to the time at which the mean arterial pressure is reached, and corresponds to an amplitude of oscillation of a certain percentage of the maximum amplitude, 85% for example.

Mean arterial pressure is therefore a quantity that is easily able to be measured using a mass-market device. Based on this measurement, systolic arterial pressure and diastolic arterial pressure may be calculated. It is common for mean arterial pressure, although measured, not to be communicated to the user, to give prominence to the systolic and diastolic arterial pressures.

The transmural pressure of an artery corresponds to a difference between the pressure inside the artery, or arterial pressure, and the pressure applied to the artery by a means for applying pressure to the artery, such as a cuff.

Pₜ=P−Pₑₓₜ (1)

When the pressure applied by the cuff corresponds to the mean arterial pressure, the transmural pressure is considered zero. The artery then has its maximum compliance. The compliance of an artery corresponds to the ratio of the variation in its cross-sectional area to the variation in transmural pressure.

### Thus:

$$C ( {p}_{t} ) = \frac{Δ A}{Δ {p}_{t}} \qquad ( 2 )$$

where:
pₜ is transmural pressure;
C(pₜ) is the compliance of the artery;
ΔA is the variation in the cross-sectional area of the artery;
Δpₜ is the variation in transmural pressure.

The cross-sectional area is the volume of blood present in a section of artery of length L, divided by said length L.

FIG. 1A schematically shows application of pressure around an artery. For a given arterial pressure, the more the pressure applied to the artery increases, the more the cross-sectional area of the artery decreases.

FIG. 1B shows the variation in the compliance of the artery (y-axis—unit mm² per mm of mercury) as a function of transmural pressure (x-axis—unit mm of mercury). It may be seen that the compliance of the artery, i.e. its elasticity, is maximum when the transmural pressure is zero, i.e. when the pressure outside the artery corresponds to the mean arterial pressure. This property is exploited in the devices for measuring mean arterial pressure described above.

In the prior art, certain developments have allowed arterial pressure to be estimated based on a measurement of a physiological parameter able to measured with a device that is more compact than cuffs equipped with pressure sensors. The physiological parameter is dependent on the compliance of the artery. It may for example be a question of an oscillation amplitude or of a transit time of a pulse wave between two detectors or of a pulse wave velocity. Such a parameter is easily measurable, for example by way of optical photoplethysmography (PPG) measurements. However, in existing methods, estimation of arterial pressure based on measurements of a physiological parameter requires a calibration phase, in the course of which measurements of the physiological parameter are taken for various arterial pressures of a user. However, this type of calibration assumes that the arterial pressure of the user is able to vary. Such a calibration is therefore time consuming, and is valid only in the range of arterial pressures of the user during the calibration phase. It is relatively easy to increase the arterial pressure of a user, for example by subjecting her or him to a stress or to physical exercise. However, it is more difficult to lower the arterial pressure of a user. It will be understood that estimation of an arterial pressure, directly based on measurements of a physiological parameter, requires a tricky calibration phase, which depends on the ability to modulate the arterial pressure of the user.

The inventors provide a method allowing an estimation of an arterial pressure based on a measurement of an easily measurable physiological parameter of a user. However, contrary to the methods described in the preceding paragraph, the method forming one subject of the invention is based on a rapid calibration that is simpler to implement, and that does not require the arterial pressure of the user to be modified. Thus, the calibration may be frequently repeated.

### SUMMARY OF THE INVENTION

A first subject of the invention is a method for determining an arterial pressure of a user, by measuring a physiological parameter, the physiological parameter passing through an extremum when the transmural pressure of the artery is zero, the method comprising the following steps:
a) applying a pressure to the artery, so as to modify the transmural pressure of the artery, the transmural pressure corresponding to a difference between the arterial pressure and the pressure applied to the artery, the applied pressure being known or measured;
b) simultaneously with step a), measuring the physiological parameter of the user by means of a sensor;
steps a) and b) being reiterated at various calibration times, while modifying the pressure applied to the artery, so that, in a step b), the extremum of the physiological parameter is measured, the applied pressure then corresponding to a mean arterial pressure of the user at the calibration times;
c) based on the pressure applied and on the physiological parameter measured in steps a) and b), establishing a calibration function, the calibration function defining a relationship between the transmural pressure and the physiological parameter;
d) after the calibration times, applying a pressure to the artery at at least one measurement time, and measuring the physiological parameter at the measurement time, the pressure applied at the measurement time being known or measured;
e) applying the calibration function resulting from c) to the physiological parameter measured at the measurement time, so as to estimate a transmural pressure at the measurement time;
f) based on the transmural pressure estimated in step e) and on the pressure applied at the measurement time, estimating an arterial pressure of the user.

Step c) is performed by a processing unit programmed to this end. The processing unit may comprise a microprocessor.

Step f) may comprise adding the transmural pressure estimated in step e) and the pressure applied in step d).

The physiological parameter may be a parameter relating to the compliance of the artery.

In step a) and/or step d), the pressure may be applied by means of a means for applying a pressure to the artery of the user, an inflatable cuff for example, configured to compress the artery.

The calibration function may be obtained by applying a regression model based on the pressure applied and on the physiological parameter measured at each calibration time.

According to one possibility:
in steps a) to b), the pressure applied to the artery varies up to a reference pressure at which the physiological parameter reaches the extremum, the reference pressure corresponding to a mean arterial pressure at the calibration times;
in step d), the pressure applied to the artery is below the reference pressure.

In step d), the pressure applied to the artery may be 50% below or 25% below the reference pressure.

According to one possibility:
various physiological parameters are taken into account in steps a) and b), so as to measure an extremum of each physiological parameter;
in step c), the calibration function determines a relationship between the transmural pressure and each physiological parameter;
step d) comprises measuring each physiological parameter at the measurement time;
in step e), the transmural pressure at the measurement time is estimated based on the calibration function determined in step c) and on the physiological parameters measured in step d).

According to one possibility, steps d) to f) are implemented at various measurement times, at a frequency higher than a heart rate of the user, so as to obtain a variation in the arterial pressure of the user between said measurement times.

In step f), the arterial pressure estimated may be a mean arterial pressure of the user.

A second subject of the invention is a device for estimating an arterial pressure of a user, comprising:
a sensor, configured to be applied facing an artery of the user, and configured to measure a physiological parameter of the user, the physiological parameter passing through an extremum when the transmural pressure of the artery is zero;
a means for applying a pressure to the artery of the user, an inflatable cuff for example, configured to apply a variable pressure to the artery of the user;
a processing unit, intended to implement steps c), e) and f) of a method according to the first subject of the invention, based on the physiological parameter measured by the sensor and on the pressure applied to the artery.

The device may comprise a pressure sensor, configured to quantify the pressure applied to the artery by the means for applying a pressure to the artery of the user.

The sensor may be chosen from: an acoustic sensor, an optical sensor, a tonometric sensor or an impedance sensor, or an electromechanical sensor. The sensor may be held around a limb of the user by a strap, the means for applying a pressure to the artery of the user being integrated into the strap or secured to the strap.

The invention will be better understood on reading the description of the examples of embodiment that are presented, in the rest of the description, with reference to the figures listed below.

### FIGURES

FIG. 1A illustrates a variation in the cross-sectional area of an artery as a function of the pressure applied to the artery.

FIG. 1B shows a variation in compliance as a function of transmural pressure.

FIGS. 2A and 2B are schematics showing the device.

FIG. 3 summarizes the main steps of to the invention.

FIG. 4 schematically shows a gradual increase in a pressure applied by a cuff.

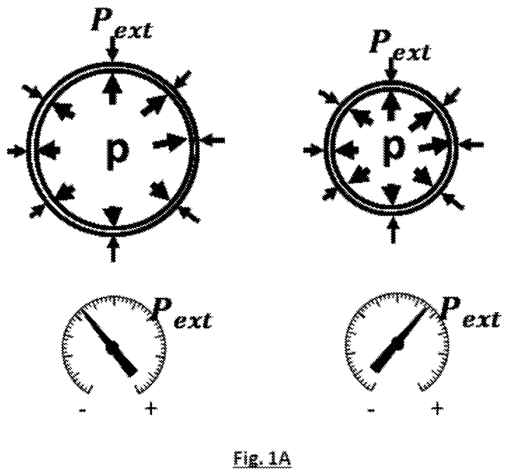

### DESCRIPTION OF PARTICULAR EMBODIMENTS

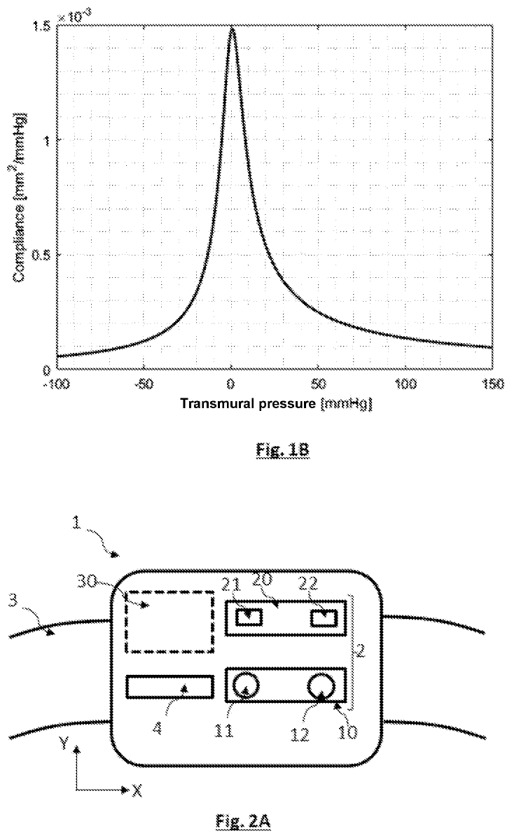

FIGS. 2A and 2B schematically show a device according to the invention. The device is intended to be worn by a user, in particular around a limb, an arm or wrist for example. The device 1 comprises a strap 3, allowing the device to be held around the limb of the user. Preferably, this strap forms an inflatable cuff. The inflatable cuff is configured to compress the limb when it is inflated. More generally, the device comprises a means for applying a pressure to an artery of the user.

The device comprises a sensor 2 configured to measure at least one physiological parameter of the user. The measured physiological parameter varies as a function of a transmural pressure of an artery of the user, facing which artery the device 1 is placed. It is notably a question of a physiological parameter related to the compliance of the artery that is liable to vary as a function of the transmural pressure of the artery.

The physiological parameter may be, non-limitingly, a pulse wave velocity, or an amplitude of oscillation of the wall of the artery.

The physiological parameter has an extreme (minimum or maximum) value when the artery is at its maximum compliance, i.e. when the pressure applied to the artery corresponds to the arterial pressure: the transmural pressure is then zero. When the physiological parameter is pulse wave velocity, it reaches a minimum value when the transmural pressure is zero. When the physiological parameter is oscillation amplitude, it reaches a maximum value when the transmural pressure is zero.

In the example shown, the sensor 2 comprises a light source 10 and a photodetector 20. The light source 10 comprises a first elementary light source 11 and a second elementary light source 12. The first and second light source are distant from each other, in a direction in which the artery extends. The distance between the first and second elementary light sources may be smaller than 5 cm. The smaller this distance, the more compact the device, to the point that it may for example be held by a strap placed around an arm of the user. The photodetector 20 comprises a first elementary photodetector 21 and a second elementary photodetector 22.

The first elementary light source 11 and the second elementary light source 12 are for example light-emitting diodes (LEDs). The first elementary light source 11 emits an incident light beam that propagates toward the tissues of the user, along a propagation axis, which is preferably perpendicular to the surface of the body of the user. The light beam is preferably emitted in a narrow spectral band, preferably<50 nm in width, in a spectral range comprised between 400 nm and 1100 nm.

The first elementary photodetector 21 and the second elementary photodetector 22 are for example photodiodes.

As known, the photons of the light beam penetrate into the tissues of the user and some thereof are backscattered in a direction parallel to the propagation axis, in a direction opposite to the latter. The backscattered photons resulting from the first elementary light source 11 are detected by the first elementary photodetector 21. The backscattered photons resulting from the second elementary light source 12 are detected by the second elementary photodetector 22.

The operating principle of the sensor 2 is based on the fact that, on each heart beat, the flow of blood in front of each elementary photodetector leads to a modulation of the absorption of light propagating through the biological tissues forming the body of the user. This results in a modulation of the intensity of the light detected by the first elementary photodetector 21 and by the second elementary photodetector 22. Thus, the intensity detected by each photodetector forms a periodic signal, the fundamental frequency of which corresponds to heart rate. The spacing between the two elementary photodetectors 21, 22 makes it possible to determine a time shift between the periodic signals delivered by the first photodetector and second photodetector, respectively. Estimation of the time shift, which is called the pulse wave transit time, allows the so-called pulse wave velocity (PWV) to be estimated by dividing the distance between the first photodetector and the second photodetector.

Alternatively, PWV may be determined via a passive measurement, for example using two mechanical or electromechanical sensors of tonometer type, spaced apart by a known distance: with this type of sensor, a deformation of the tissue under the effect of the pulse wave is measured, without an excitation signal.

Other methods for measuring the physiological parameter may be based on electrical excitation of the tissues, and on a measurement of a response, which is also electrical, of the tissues, according to the principle of measurements of electrical impedance. Under the effect of a periodic variation in the volume of blood, the electrical impedance varies periodically. Thus, a measurement of electrical impedance allows cardiac activity to be monitored. The shift between the periodic variations in electrical impedance allows pulse wave velocity to be estimated. This type of modality is described in the publication by Bassem I. et al “Multi-source multi-frequency bio-impedance measurement method for localized pulse wave monitoring”, 2020. A measurement of pulse wave velocity may also be obtained using an acoustic method, by determining a periodic variation in acoustic tissue impedance induced by cardiac activity. The principle is then based on emission of an acoustic wave that propagates to the tissues, and on detection of an acoustic wave reflected by the tissues. The sensor 2 may comprise two elementary acoustic sensors distant from each other, the distance between the two sensors being known. Each elementary sensor allows a periodic variation in acoustic impedance to be determined. The shift between the periodic variations respectively determined by each elementary sensor allows pulse wave velocity to be estimated. This type of modality is described in the publication by Hermeling E et al. “The dicrotic notch as alternative time-reference point to measure local pulse wave velocity in the carotid artery by means of ultrasonography”, Journal of hypertension, 2009, 2028-2035.

The physiological parameter may also be measured by a single sensor, a single photodetector for example. This is for example the case when the measured physiological parameter is a vibration amplitude of the pulse wave. Such a parameter is measurable with a PPG sensor.

Generally, the one or more sensors are configured to form a spike under the effect of the pulse wave. The physiological parameter is determined depending on an area or height of the spike (for example when the vibration amplitude of the pulse wave is being measured), or based on a time shift between two spikes measured by two sensors spaced apart from each other (for example when pulse wave velocity is being measured).

The device may also comprise a pressure sensor 4, configured to determine the pressure with which the device is pressed against the user. The data delivered by the pressure sensor 4 are transmitted to the processing unit 30.

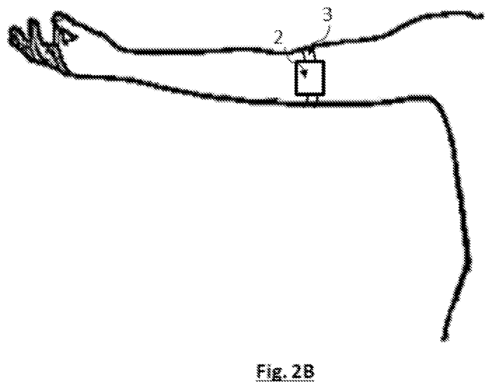

The device 1 comprises a processing unit 30. The processing unit is connected to the photodetectors 21 and 22, so as to measure pulse wave velocity. The processing unit 30 is programmed to implement the steps illustrated in FIG. 3. The processing unit may notably comprise a microprocessor or a microcontroller.

Steps 91 to 93 correspond to a calibration phase 90. In step 91, the cuff 3 is inflated, so as to make the pressure applied to the artery vary. This allows the transmural pressure to be varied. In this example, pressure is gradually increased, this leading to a gradual decrease in transmural pressure. In step 92, PWV (or any other physiological parameter that varies under the effect of a variation in transmural pressure) is measured.

Steps 91 and 92 are reiterated, while gradually increasing (or decreasing) the pressure applied to the artery.

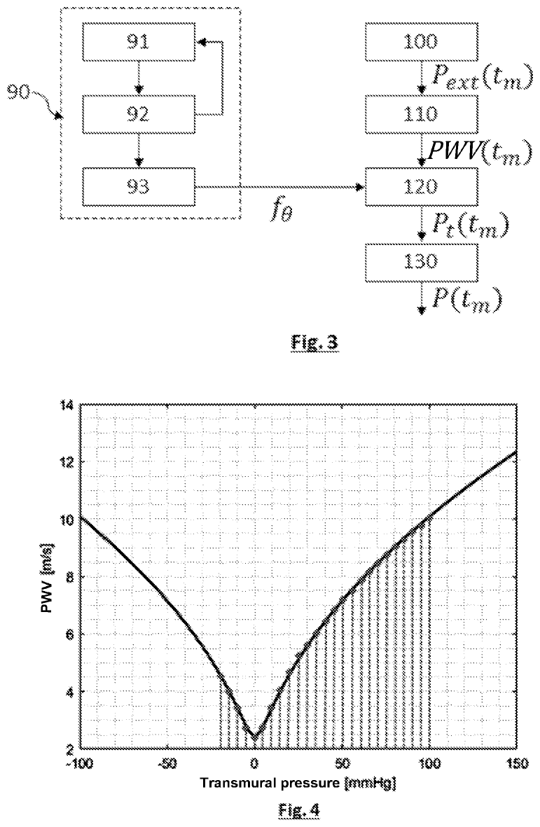

FIG. 4 schematically shows a gradual increase in applied pressure (or external pressure), resulting in a gradual decrease in transmural pressure. Each vertical line corresponds to one measurement point, at one calibration time. At each calibration time, pulse wave velocity is measured by the device. The gradual decrease in transmural pressure leads to a decrease in pulse wave velocity, until a minimum pulse-wave-velocity value is reached. The minimum pulse-wave-velocity value is reached when the transmural pressure is zero, i.e. when the pressure exerted on the artery corresponds to the mean arterial pressure. When the applied pressure is further increased, transmural pressure becomes negative and pulse wave velocity gradually increases again. The iterations of steps 91 and 92 are then stopped, because the minimum pulse wave velocity has been reached.

The minimum PWV value is obtained by applying a pressure corresponding to what is called a reference pressure. At the reference pressure P_{ext,ref}, the transmural pressure of the artery is considered to be zero. The reference pressure corresponds to the mean arterial pressure of the user during the calibration phase.

At the end of steps 91 and 92, pairs of values of the PWV and external pressure measured at each calibration time are obtained. Thus, if t_{c} corresponds to a calibration time, at each calibration time pairs (Pₑₓₜ(t_{c}), PWV (t_{c})) are obtained, where Pₑₓₜ(t_{c}) is the external pressure applied at a calibration time. The term “external pressure” designates the pressure applied to the artery. The reference pressure P_{ext,ref}, at which the pulse wave velocity is minimum, is also known.

Based on the pairs (Pₑₓₜ(t_{c}), PWV(t_{c})) and on the value of the reference pressure P_{ref}, step 93 consists in determining a calibration function describing the variation in the transmural function Pₜ as a function of pulse wave velocity.

Pₜ(t_{c})=P_{ext,ref}−Pₑₓₜ(t_{c})=f_{θ}(PWV(t_{c})) (3)

The calibration function f_{θ}, parametrized by a set of parameters θ, may be analytical, for example a polynomial function inter alia. It may also be a function determined by a neural network or, more generally, an artificial-intelligence supervised-learning algorithm. The calibration function may be determined by applying a regression model to the pairs (Pₜ(t_{c}), PWV(t_{c})) resulting from steps 91 and 92. It may for example be a linear regression.

In the calibration phase, mean arterial pressure is assumed to remain constant. Thus, the calibration phase is preferably carried out in a short time interval, for example a few tens of seconds or a few minutes.

Steps 100 to 130 are performed at measurement times tₘ, subsequent to the calibration phase. In a step 100, the cuff applies an external pressure Pₑₓₜ(tₘ) to the artery, the external pressure being measured by the sensor 4.

Preferably, in contrast to the prior art, the applied pressure is strictly below the reference pressure P_{ext,ref}. It is preferably 50% below or even 25% below the reference pressure P_{ext,ref}. It may for example be equal to 50 mm of mercury. The applied pressure is above a minimum pressure, which is determined on a case-by-case basis, depending on the device employed.

In prior-art devices, employing a cuff coupled to a pressure sensor, measurement of mean arterial pressure requires the cuff to be inflated beyond the point where the maximum compliance of the artery is reached. The pressure exerted on the artery reaches and then exceeds the mean arterial pressure. If a measurement of mean arterial pressure must be taken repeatedly, the user experiences a certain amount of discomfort as a result, because of the pressure level applied. In addition, it is incompatible with use while the user is sleeping. Applying a measurement pressure below the reference pressure P_{ext,ref} increases the comfort of the user with respect to prior-art devices, in which the applied pressure reaches or exceeds arterial pressure. Thus, arterial pressure may be measured while limiting the discomfort caused to the user.

In step 110, the PWV at the measurement time, which is denoted PWV(tₘ), is measured by the sensor 2.

In step 120, the transmural pressure is estimated from the calibration function.

Pₜ(tₘ)=f_{θ}(PWV(tₘ)) (4)

In step 120, the arterial pressure, at the measurement time, is estimated using the expression:

P(tₘ)=Pₜ(tₘ)+Pₑₓₜ(tₘ) (5)

Steps 100 to 130 may be implemented at various measurement times.

Equations (4) and (5) are a key aspect of the invention. In contrast to the prior art, the calibration function makes it possible to estimate, based on the measured physiological parameter, the transmural pressure rather than the arterial pressure. Such a calibration function is much easier to obtain, since it is enough to vary the pressure applied to the artery and to determine the reference pressure, at which the transmural pressure is considered to be zero. In contrast to the prior art, the calibration does not require the arterial pressure of the user to be made to vary. On the contrary, the arterial pressure is preferably considered to remain constant during the calibration. This is justified by the fact that the calibration is quick to perform: it is enough to vary the pressure up to or from the reference pressure, at which the physiological parameter reaches an extreme (maximum or minimum) value. When the extreme value is reached, the applied external pressure is considered to correspond to the mean arterial pressure of the user.

Based on the transmural pressure, estimated by applying the calibration function to the measured physiological function (equation 4), the arterial pressure is obtained in a straightforward manner, simply by adding the estimated transmural pressure Pₜ(tₘ) and the applied external pressure Pₑₓₜ(tₘ) (equation 5). The applied pressure is either measured, or pre-set and therefore known.

Apart from the parameters θ determined by the regression, the calibration function may take into account anthropometric parameters, for example size, weight, gender or age.

The pressure P(tₘ) estimated using expression (5) corresponds to an arterial pressure at the measurement time tₘ. When the pressure Pₑₓₜ(tₘ) is measured by a pressure sensor having a long response time, longer than several hundred ms or than 1 second, the pressure P(tₘ) corresponds to a mean arterial pressure over one or more beats. Specifically, on account of the long response time, the pressure Pₑₓₜ(tₘ) corresponds to a mean applied pressure.

When the pressure Pₑₓₜ(tₘ) is measured by a pressure sensor having a short response time, for example of about a few tens of ms, the pressure P(tₘ) corresponds to a “real time” arterial pressure, at the measurement time tₘ. The pressure P(tₘ) takes into account the variation in the arterial pressure between two consecutive beats. A component of the arterial pressure, which is referred to as the pulsed component, is thus accessed. Depending on the measurement times, the systolic pressure (maximum pressure within a given cycle) or diastolic pressure (minimum pressure within the same cycle) may be obtained. In this case, the measurement times are spaced apart such as to obtain a frequency higher than the heart rate of the user, for example 5 or 10 times higher. It is then possible to estimate the variation in the transmural pressure between two successive beats. This allows the variation in the arterial pressure between two successive beats to be obtained.

As indicated above, the invention may be implemented with another physiological parameter. It may for example be a question of the amplitude of oscillation of the artery. In this case, in the calibration phase, the pressure is gradually increased until a maximum vibration amplitude is reached. Then only a single source-detector pair is required, for example the first elementary source 11 and the first elementary photodetector 21. When the maximum amplitude is reached, the applied pressure corresponds to the reference pressure, i.e. to the mean arterial pressure of the user during the calibration phase.

Other examples of exploitable physiological parameters are given below:
pulse transit time, i.e. the transit time of the pulse wave between two measurement times, usually designated by the acronym PWTT (standing for pulse wave transit time);
time between the systolic peak and the diastolic peak of a given beat;
areas of the systolic peak and diastolic peak such as measured by the detector.

Thus, generally, the sensor is configured to obtain a pulse under the effect of a pulse wave: it may, by way of non-limiting example, be a question of an optical sensor, an acoustic sensor, an electrical sensor, or an electromechanical sensor (accelerometer).

The calibration phase may be repeated periodically, so as to update the calibration function. The update may be performed regularly, weekly for example.

According to one possibility, a plurality of physiological parameters may be used to determine the arterial pressure. Each physiological parameter reaches an extreme (minimum or maximum) value when the transmural pressure is zero. In this case, the calibration function is multi-parametric, in the sense that it depends on each physiological parameter considered. The calibration is performed as described above,
by increasing the pressure applied to the artery to a reference pressure, at which each parameter is considered to have reached its maximum value or its minimum value;
and/or by decreasing the pressure applied to the artery from a reference pressure, at which each parameter is considered to have reached its maximum value or its minimum value.

Based on the various values of the parameters measured at each applied pressure, and based on the reference pressure, at which the transmural pressure is considered to be zero, the calibration function is determined, for example by regression.

The invention allows the arterial pressure of the user to be measured frequently, at various times during the same day, while limiting the discomfort of the user. This makes it particularly suitable for users requiring their arterial pressure to be regularly monitored.

1. A method for determining an arterial pressure of a user, by measuring a physiological parameter, the physiological parameter passing through an extremum when a transmural pressure of the artery is zero, the method comprising:
a) applying a pressure to the artery, so as to modify the transmural pressure of the artery, the transmural pressure corresponding to a difference between the arterial pressure and the pressure applied to the artery, the applied pressure being known or measured;
b) simultaneously with step a), measuring the physiological parameter of the user by means of a sensor;
steps a) and b) being reiterated at various calibration times, while modifying the pressure applied to the artery, so that, in the course of one iteration, a step b) comprises applying a reference pressure at which the extremum of the physiological parameter is measured, the reference pressure that corresponding to the mean arterial pressure of the user at the calibration times;
c) based
on the pressure applied and on the physiological parameter measured at each calibration time;
and on the reference pressure;
determining the transmural pressure at each calibration time and establishing a calibration function, the calibration function defining a relationship between the transmural pressure and the physiological parameter;
d) after the calibration times, applying a pressure to the artery at at least one measurement time, and measuring the physiological parameter at the measurement time, the pressure applied at the measurement time being known or measured;
e) applying the calibration function resulting from c) to the physiological parameter measured at the measurement time, so as to estimate a transmural pressure at the measurement time;
f) based on the transmural pressure estimated in e) and on the pressure applied at the measurement time, estimating an arterial pressure of the user.

2. Method according to claim 1, wherein step f) comprises adding the transmural pressure estimated in step e) and the pressure applied in step d).

3. Method according to claim 1, wherein the physiological parameter is a parameter relating to the compliance of the artery.

4. Method according to claim 1, wherein, in step a) or step d), the pressure is applied by means of a means for applying a pressure to the artery of the user, configured to compress the artery.

5. Method according to claim 1, wherein the calibration function is obtained by applying a regression model based on the pressure applied and on the physiological parameter measured at each calibration time.

6. Method according to claim 1, wherein in step d), the pressure applied to the artery is below the reference pressure.

7. Method according to claim 6, wherein, in step d), the pressure applied to the artery is 50% below or 25% below the reference pressure.

8. Method according to claim 1, wherein:
various physiological parameters are taken into account in steps a) and b), so as to measure an extremum of each physiological parameter;
in step c), the calibration function determines a relationship between the transmural pressure and each physiological parameter;
step d) comprises measuring each physiological parameter at the measurement time;
in step e), the transmural pressure at the measurement time is estimated based on the calibration function determined in step c) and on the physiological parameters measured in step d).

9. Method according to claim 1, wherein steps d) to f) are implemented at various measurement times, at a frequency higher than a heart rate of the user, so as to obtain a variation in the arterial pressure of the user between said measurement times.

10. Method according to claim 1, wherein, in step f), the arterial pressure estimated is a mean arterial pressure of the user.

11. Device for estimating an arterial pressure of a user, comprising:
a sensor, configured to be applied facing an artery of the user, and configured to measure a physiological parameter of the user, the physiological parameter passing through an extremum when a transmural pressure of the artery is zero;
a means for applying a pressure to the artery of the user, configured to apply a variable pressure to the artery of the user;
a processing unit, intended to implement steps c), e) and f) of a method according to claim 1 based on the physiological parameter measured by the sensor and on the pressure applied to the artery.

12. Device according to claim 11, comprising a pressure sensor, configured to quantify the pressure applied to the artery by the means for applying a pressure to the artery of the user.

13. Device according to claim 11, wherein the sensor is chosen from: an acoustic sensor, an optical sensor, a tonometric sensor or an impedance sensor, or an electromechanical sensor.

14. Device according to claim 11, wherein the sensor is held around a limb of the user by a strap, the means for applying a pressure to the artery of the user being integrated into the strap or secured to the strap.
